(19) United States
(12) Patent Application Publication
(10) Pub. No.: US 2013/0292870 A1
(43) Pub. Date: Nov. 7, 2013

(54) METHODS FOR MANUFACTURING CUSTOM CUTTING GUIDES IN ORTHOPEDIC APPLICATIONS

(71) Applicant: Howmedica Osteonics Corp., Mahwah, NJ (US)

(72) Inventor: Christopher Abee Roger, Waldwick, NJ (US)

(21) Appl. No.: 13/934,792

(22) Filed: Jul. 3, 2013

Related U.S. Application Data
(63) Continuation of application No. 12/541,443, filed on Aug. 14, 2009, now abandoned.

(51) Int. Cl. A61B 17/15; B29C 41/02

(52) U.S. Cl. CPC A61B 17/155; B29C 41/02
USPC 264/138; 264/219

(57) ABSTRACT

A patient specific system for joint replacement surgery that includes a custom cutting guide having an inner surface shaped to match the anatomy of a surface of a patient's joint to be resected. The custom cutting guide is designed for use with a corresponding prosthesis. A slot and guide holes are formed in the custom cutting guide corresponding to features protruding outwardly from a positive physical bone model. The slot guides a tool during resection of the femur to produce a first resected surface on the femur for mounting the prosthesis. The guide is formed from the positive physical model by applying a polymeric composition to the outer surface of the positive physical model including the features corresponding to the slot and guide holes of the custom cutting guide.

### CROSS-REFERENCE TO RELATED APPLICATIONS

The present application is a continuation of U.S. patent application Ser. No. 12/541,443, filed Aug. 14, 2009, the disclosure of which is incorporated herein by reference in its entirety.

### FIELD OF THE TECHNOLOGY

The present invention relates to creating a patient specific cutting guide from a positive physical model of a surface of a patient's joint, the model including features corresponding to features of the guide. In particular, the present invention relates to determining the location and orientation of a cutting plane virtually, such as a distal cutting plane in a distal femoral resection, creating the positive physical model substantially replicating the virtual model, and creating the cutting guide from the positive physical model.

### BACKGROUND OF THE INVENTION

Joint replacement procedures are used to repair damaged joints. During a joint replacement procedure the joint is preferably aligned, bone or bones of the joint may be resected, and a prosthesis may be implanted on the resected bone. Joint replacement procedures may be performed on the knee, hip, shoulder or elbow joints, for example. Accuracy of joint alignment and bone resection is crucial in a joint replacement procedure. A small misalignment may result in ligament imbalance and consequent failure of the joint replacement procedure. Provision of patient specific or customized cutting guides and prostheses can improve the outcome of joint replacement procedures.

U.S. Pat. No. 8,092,465 (“the '465 Patent”) teaches a method of preparing a joint for a prosthesis in a patient. The method includes obtaining scan data associated with the joint of the patient, preparing a three-dimensional image of the joint based on the scan data, preparing an interactive initial surgical plan based on the scan data, sending the surgical plan to a surgeon, receiving a finalized surgical plan from the surgeon, and preparing an image of a patient-specific alignment guide. The patient specific alignment guide of the '465 Patent includes an inner guide surface designed to closely conform, mate and match the femoral joint surface of the patient in three-dimensional space such that the alignment guide and the femoral joint surface are in a nesting relationship to one another. Accordingly, the alignment guide can conform, mate and snap on or “lock” onto the distal surface of the femur in a unique position determined in the final surgical plan. Apertures in the alignment guide may be used to locate a femoral resection block or other cutting device in the distal femur.

Other custom guides for femoral resections are known to have a distal cutting slot formed therein, the custom guide having an inner guide surface designed to conform to the femoral joint surface. Such guides are generally manufactured with guide holes and the distal cutting slot in a position to achieve a desired distal cut such that a 4-in-1 cutting block may then be easily placed on the resected distal surface of the femur. After the femur is resected using the custom guide and the 4-in-1 cutting block, a femoral prosthesis may be implanted on the resected femur.

International Publication Number WO 93/25157, for example, discloses a template that has parts of a surface of an arbitrary osseous structure which is to be treated and is intraoperatively accessible to the surgeon, copied as a negative image without undercut and in a mechanically rigid manner, so that the individual template can be set onto the osseous structure in a clearly defined position and with mating engagement. In the context of spinal surgery, WO 93/25157 discloses making a template having contact faces so that the template can be set directly onto the exposed bone surface, including any surrounding tissue in a clearly defined manner. Having contact faces on the template for use in spinal surgery instead of providing the template with a surface that is negative image of spine makes sense because of the highly complex shape of the spine.

WO 93/25157, when addressing the hip joint, discloses template that has large area that is negative image without undercut and in a mechanically rigid manner, so that the individual template can be set onto the osseous structure in a clearly defined position and with mating engagement.

In each of the above described guides and methods of creating or using the same, the position of guide holes and cutting slot of a custom guide is generally planned virtually and thereafter manufactured through various method such as injection molding, selective laser sintering (SLA), or casting, for example.

### SUMMARY OF THE INVENTION

A first aspect of the present invention is a method of creating a patient specific cutting guide. According to this first aspect the method preferably includes creating a virtual model of a patient's bone to be resected using the patient specific cutting guide. The method preferably includes determining at least one resection on the virtual model and creating an updated virtual model including the at least one resection plane. Preferably, the method includes creating a physical model of the patient's bone from the updated virtual model and covering at least a portion of the physical model with a curable polymeric composition. The method preferably further includes allowing the polymeric composition to harden to form the patient specific cutting guide, wherein the patient specific cutting guide includes a reference location defining the at least one resection plane for guiding a cutting tool.

A second aspect of the present invention is a method of creating a patient specific femoral cutting guide. According to this second aspect, the method preferably includes obtaining data defining the geometry of a patient's femur to create a virtual model of at least a portion of the patient's femur. The method preferably includes determining at least one set of two first reference locations on the virtual model each representing the location of a guide hole on the patient specific cutting guide and determining a reference plane that extends outwardly from the virtual model. Preferably, the method includes creating an updated virtual model by adding to the virtual model at least two protrusions extending outwardly from the virtual model and adding to the virtual model a thin wall extending in an anterior direction from the reference plane. The method preferably further includes creating a physical model of the updated virtual model, covering the physical model with a polymeric composition, and allowing the polymeric composition to harden to form the patient specific cutting guide.

A third aspect of the present invention is a physical model of a patient's bone for creating a patient-specific cutting guide. According to this third aspect, the physical model preferably includes an exterior surface defining the external geometry of the patient's bone, a first set of two posts protruding from the exterior surface of the model, the first set of two posts representing the location and approximate size of a first set of guide holes on the patient specific cutting guide, and a wall protruding from the model, the wall representing the location of a cutting slot on the patient specific cutting guide.

A fourth aspect of the present invention is a patient specific cutting guide conforming to a patient's bone. According to this fourth aspect, the patient specific cutting guide includes a hardened polymeric composition including an inner surface having at least three contact points with an exterior surface of a patient's bone, the hardened polymeric composition having at least a first set of two guide holes and a cutting slot formed therein, the hardened polymeric composition forming a negative model from a positive physical model of the patient's bone, the positive physical model having at least first set of two posts protruding from an exterior surface of the model, the first set of two posts representing the location and diameter of the first set of two guide holes of the hardened polymeric composition, and a wall protruding from the exterior surface of the model, the wall representing the location of a cutting slot on the hardened polymeric composition.

### BRIEF DESCRIPTION OF THE DRAWINGS

FIG. 1 is an isometric view of an embodiment of a positive physical bone model of the present invention, including features corresponding to features of a custom cutting guide.

FIG. 2 is an alternative isometric view of the positive physical model of FIG. 1 attached to a drill bit.

FIG. 3 shows the positive physical model of FIG. 1 attached to a lathe.

FIG. 4 shows a parting film being applied to the exterior surface of the positive physical model of FIG. 1.

FIG. 5 shows a polymeric composition being applied to the exterior surface of the positive physical model of FIG. 1.

FIG. 6 shows the outside surface of the positive physical model of FIG. 1 covered with the polymeric composition.

FIG. 7 shows the cutting of the features of the positive physical model along the exterior surface of the cured polymeric composition which has formed a custom cutting guide.

FIG. 8 shows a tangent line being drawn on the exterior surface of the custom cutting guide.

FIG. 9 shows the custom cutting guide being cut along the tangent line thereof such that the guide may be removed from the positive physical model.

FIG. 10 is an isometric view of an embodiment of a custom cutting guide of the present invention, including features in the form of guide holes and a distal cutting slot corresponding to features of the positive physical model of FIG. 1.

FIG. 11 is a view of an inner surface of the custom cutting guide shown in FIG. 10.

FIG. 12 is a view of the custom cutting guide shown in FIG. 10. before it is attached to the exposed outer surface of a distal femur.

FIG. 13 is a view of the custom cutting guide shown in FIG. 10. attached to the exposed outer surface of the distal femur shown in FIG. 12.

FIG. 14 is a top plan view of the custom cutting guide shown in FIG. 10 attached to the exposed outer surface of the distal femur shown in FIG. 12.

FIG. 15 is an isometric view of a resected distal surface of the distal femur shown in FIG. 12.

FIG. 16 is a view of a four-in-one cutting block before it is attached to the resected distal surface of the distal femur shown in FIG. 12.

FIG. 17 is a view of the four-in-one cutting block shown in FIG. 16 attached to the resected distal surface of the distal femur shown in FIG. 12.

FIG. 18 is a view of the distal femur shown in FIG. 12 having been resected by the custom cutting guide and the four-in-one cutting block.

FIG. 19 is a view of a prosthesis before it is attached to the resected distal surface of the distal femur shown in FIG. 12.

### DETAILED DESCRIPTION

As used herein, when referring to bones or other parts of the body, the term “proximal” means closer to the heart and the term “distal” means more distant from the heart. The term “inferior” means toward the feet and the term “superior” means toward the head. The term “anterior” means toward the front part of the body or the face and the term “posterior” means toward the back of the body. The term “medial” means toward the midline of the body and the term “lateral” means away from the midline of the body.

The methods described herein generally include creating a custom cutting guide from a physical bone model having features corresponding to features of the custom cutting guide for use in orthopedic applications. A virtual model of a patient's bone or bones in a particular joint of the patient is created and then an updated virtual model including the features corresponding to the features of the custom cutting guide are created virtually as well. The physical bone model is created from the updated virtual model.

The features of the physical bone model are preferably in the form of circular posts and a wall protruding outwardly from the exterior surface of the physical bone model. These features correspond to the features in the form of guide holes and a cutting slot on the custom cutting guide. The diameter of the circular posts preferably relate to the diameter of a guide hole or a guide pin, while the length and width of the wall preferably relate to the length and width of a cutting slot of the custom cutting guide.

In one embodiment of the present invention, the method preferably includes taking a computer tomography (CT) scan or magnetic resonance imaging (MRI) of a patient's joint, for example. Other means known in the art may be used to obtain information relating to the structure of a patient's joint. In the present embodiment, the method includes taking a CT scan or MRI of a patient's knee joint, but it should be understood that the invention may be used for creating custom cutting guides for other joints, such as the hip, shoulder, or spine, for example.

In the present embodiment, data obtained from the CT scan or MRI is preferably converted to a working computer aided design (CAD) model or virtual model of the patient's joint. The conversion of the data obtained from the CT scan or MRI to a working CAD model may be done in any known manner in the art. After the CAD or virtual model of the patient's joint is created, the topography or outer surface of the bones in the joint may be visualized on a computer screen or any like visual medium. Preferably, the virtual model of the patient's joint is a three-dimensional model that may be rotated and manipulated in three-dimensions such that an operator visualizing the model on a computer screen may be able to see all structures of bones individually or of all the bones in the joint at once, such as the femur, tibia and patella in a knee joint, for example.

Determining the correct location and orientation of the distal resection plane on the virtual model is needed in order to create an accurate updated virtual model, an accurate physical bone model, and an accurate custom cutting guide from the physical bone model. Distinct anatomical landmarks may be identified on the virtual model and used as reference points for determining the location and orientation of the distal resection plane of the femur. Such anatomical landmarks on the femur may include the medial or lateral epicondyles, the medial or lateral condyles, the trochlear groove, or the intercondylar notch, for example, among other distinct anatomical landmarks.

Preferably, a virtual bone model of the patient's entire femur is created for determining where the distal resection plane should be located and oriented for a total knee arthroplasty (TKA) procedure. In one embodiment, the femoral mechanical axis of the patient may be used to determine the location and orientation of the distal resection plane in a TKA. In other embodiments, the anatomical axis of the patient may be used to determine the location and orientation of the distal resection plane in a TKA. The distal resection plane is preferably perpendicular to the femoral mechanical axis. In one embodiment, the femoral mechanical axis of a particular patient's femur may be determined by locating the center of the femoral head and the center of the hip on the virtual bone model. In another embodiment, for example, segments of the virtual bone model may be used to determine the femoral mechanical axis. The line connecting these two centers preferably represents the femoral mechanical axis. Once this axis is obtained, the surgeon may then manipulate along the axis a virtual plane oriented perpendicularly to the axis until he or she decides based on their experience or through the use of any of the other above mentioned anatomical landmarks, for example, where the plane should be located in order to obtain a correction, such as an alignment correction of the bones of the joint being operated on.

The surgeon may also manipulate a virtual model of a prosthesis to determine whether the distal resection plane is located in an optimal position because the distal surface of the prosthesis will be aligned with the distal resection plane and the surgeon will then have the opportunity to see how the orientation of the prosthesis in relation to the surrounding bone or bones of the joint. The surgeon may also manipulate differently sized prostheses on the virtual model from a library of stored virtual prostheses to determine which prosthesis is optimal to correct the alignment of a patient's joint. The surgeon will also be able to see the bone of the femur that will have to be resected in order to accommodate the anterior surface, the anterior chamfer surface, the posterior surface and the posterior chamfer surface of the selected prosthesis.

The distal resection plane represents the location and orientation of the cutting slot of the cutting guide configured to direct a cutting saw or any other like bone resection tool to remove any bone located distally of the distal resection plane of the femur. Once the correct location and orientation of the distal resection plane is identified and a prosthesis is selected, an extrusion or protrusion in the shape of a thin wall is created on the virtual bone model extending a certain distance anteriorly from the virtual bone model, representing the height of the wall, and extending distally a certain distance from the distal resection plane, representing the width of the wall.

The dimensions of the thin wall preferably represent the size of the distal cutting slot of the custom cutting guide. The dimensions of the thin wall, for example, may be approximately 0.5 mm to 6 mm in width (representing the width of the cutting slot), approximately 5 cm to 10 cm in length (representing the length of the cutting slot), and approximately 1 cm to 10 cm in height.

A plurality of preferably circular extrusions or protrusions are also created on the virtual bone model. The circular protrusions preferably extend outwardly from the virtual bone model along either an axis that is generally parallel or perpendicular to the distal resection plane. The dimensions of the plurality of protrusions, for example, may be approximately 1 mm to 20 mm in diameter (representing the approximate diameter of guide holes in the cutting guide or fixation pinholes for the 4-in-1 cutting block or holes in the distal resected surface of the femur to accommodate the fixation pins of a prosthesis that will later be implanted on the resected femur), and approximately 1 cm to 5 cm in height. The addition of the thin wall and plurality of protrusions to the virtual bone model creates the updated virtual model from which the physical bone model is created. A thin metallic material may be placed around the circumference of the posts and thin wall in order to create a more rigid guide hole or cutting slot after the polymeric composition is added to the exterior surface of the positive physical model including the posts and thin wall.

Once the updated virtual model is created, a file including all of the information from the updated virtual model is then exported into a file format that is recognized by additive manufacturing equipment. The physical bone model including the features corresponding to the features of the custom cutting guide may then be created by an additive manufacturing process such as selective laser sintering (SLS), for example.

Referring to the drawings, wherein like reference numerals represent like elements, there is shown in the figures, in according with embodiments of the present invention, a physical bone model used to create a custom cutting guide, designated generally by reference numeral 10. The custom guide preferably includes a plurality of guide holes that correspond to the plurality of protrusions on the physical bone model. Guide holes of the custom cutting guide that are positioned in a generally parallel orientation with respect to the cutting slot of the custom cutting guide are used to ensure that the guide after being attached on the femur remains engaged to the femur when the distal cut of the femur is being made. Guide holes of the custom cutting guide that are positioned in a generally perpendicular orientation with respect to the cutting slot of the custom cutting guide are used as drill guide holes that generally represent the location of the guide pins in the anterior-posterior plane for a 4-in-1 cutting block. A drill may be guided by the guide holes into a specific location on the distal resected surface of the femur. This specific location may correspond to the location of the fixation posts protruding from the distal surface of the selected prosthesis, for example.

As shown in FIG. 1, physical bone model 10 is designed to be used in creating a custom cutting guide for the distal resection of a patient's femur. Physical bone model 10 includes a thin wall 20 extending outwardly therefrom in an anterior direction. Wall 20 preferably represents a distal cutting slot of the custom cutting guide. As shown, wall 20 preferably protrudes outwardly from an exterior surface of physical bone model 10. Wall 20 also extends in a distal direction from a plane 34 adjacent and parallel to a proximal surface 22 of wall 20. A first set of two pins 30, 32 preferably extend outwardly in an anterior direction from the exterior surface of bone model 10. A second set of two pins 36, 38 preferably extend outwardly in a distal direction from the exterior surface of bone model 10 in a direction generally perpendicular to first set of two pins 30, 32 and wall 20. First set of two pins 30,32 represents the location of fixation pins (not shown) that may be used for maintaining the position of the custom guide with respect to the femur as the distal resection cut is being made using the custom cutting guide. Second set of two pins 36, 38 are used to form holes in custom cutting guide that will be used as drill guides for a drill used to create holes to house fixation pins of the four in one cutting block that will be used to make the anterior cut, anterior chamfer cut, posterior chamfer cut, and posterior cut. The holes made by the drill may also be used to house fixation posts of the prosthesis that will be implanted on the resected femur.

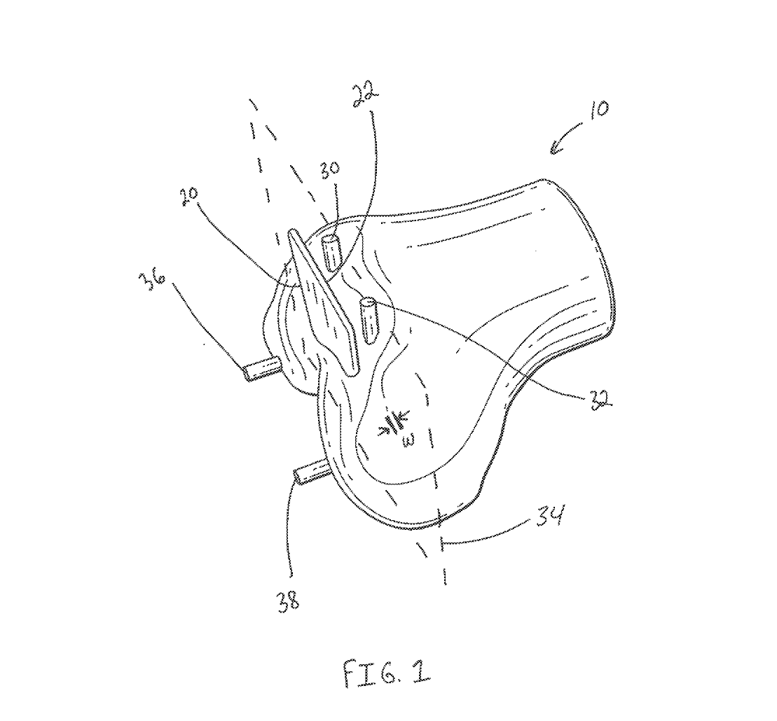

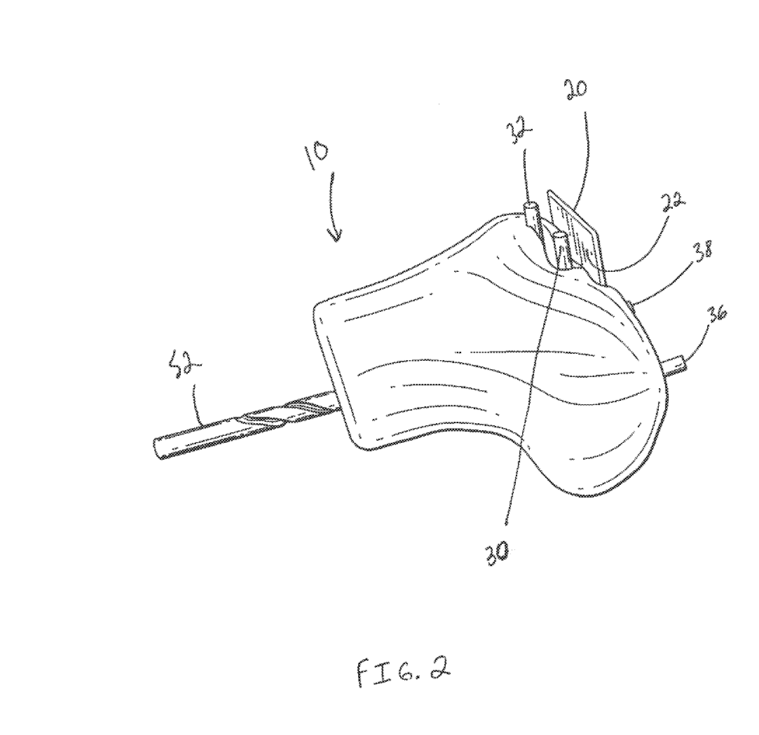

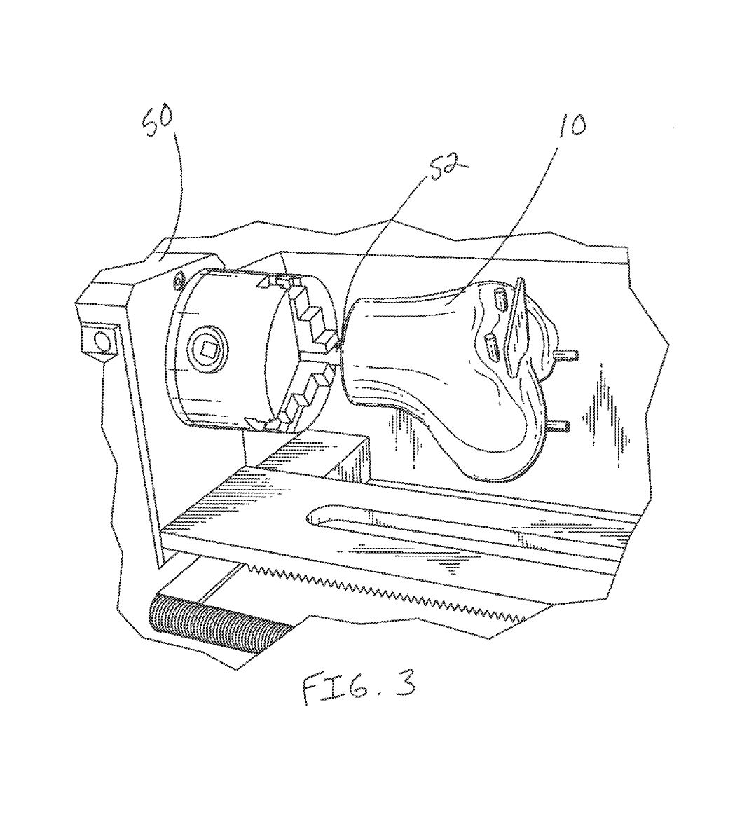

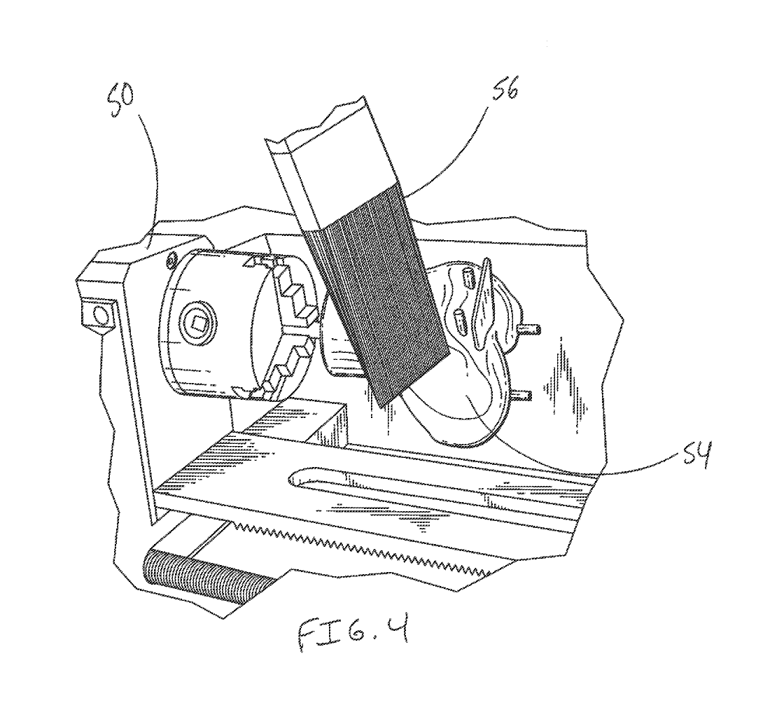

After bone model 10 is created, including wall 20, first sets of two pins 30, 32 and second set of two pins 36, 38, the custom cutting guide is preferably formed from model 10. A polymeric composition is preferably applied to an exterior surface of model 10. The polymeric composition may be applied through a spraying technique or dipped in a polymeric bath, for example. In one embodiment of forming a custom cutting guide by applying a polymeric composition to an exterior surface of model 10, a drill bit 52 may be attached to model 10 and then secured to a lathe 50 as shown in FIGS. 2 and 3. A parting film 54, for example, may be thoroughly applied by a brush 56 as shown in FIG. 4 to the exterior surface of bone model 10, including wall 20, first sets of two pins 30,32 and second set of two pins 36, 38 while the lathe rotates bone model 10.

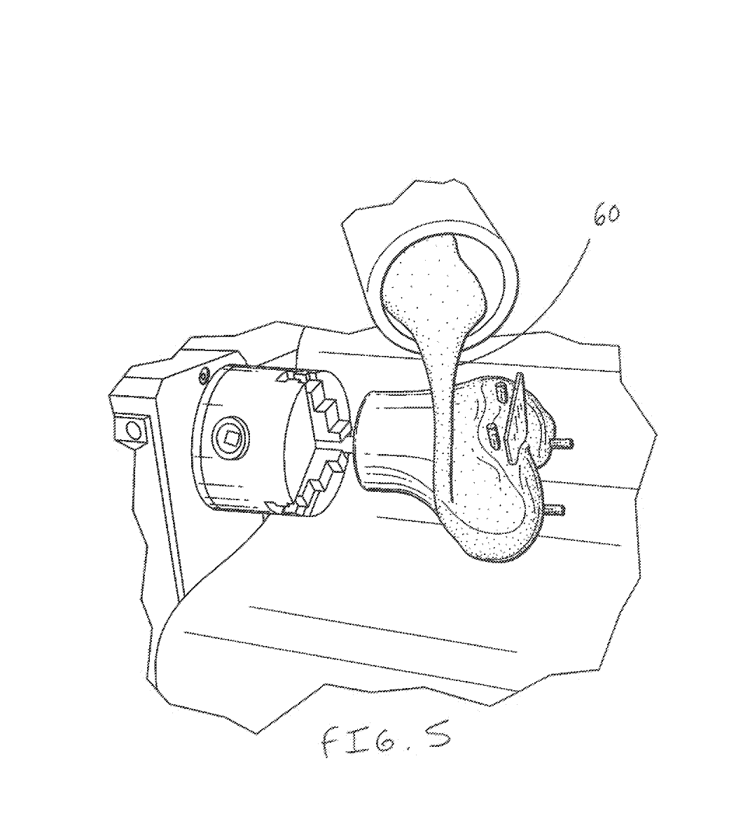

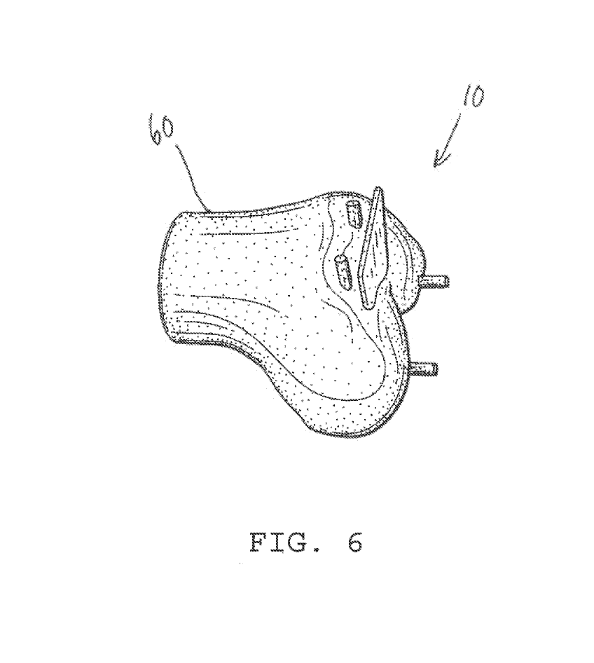

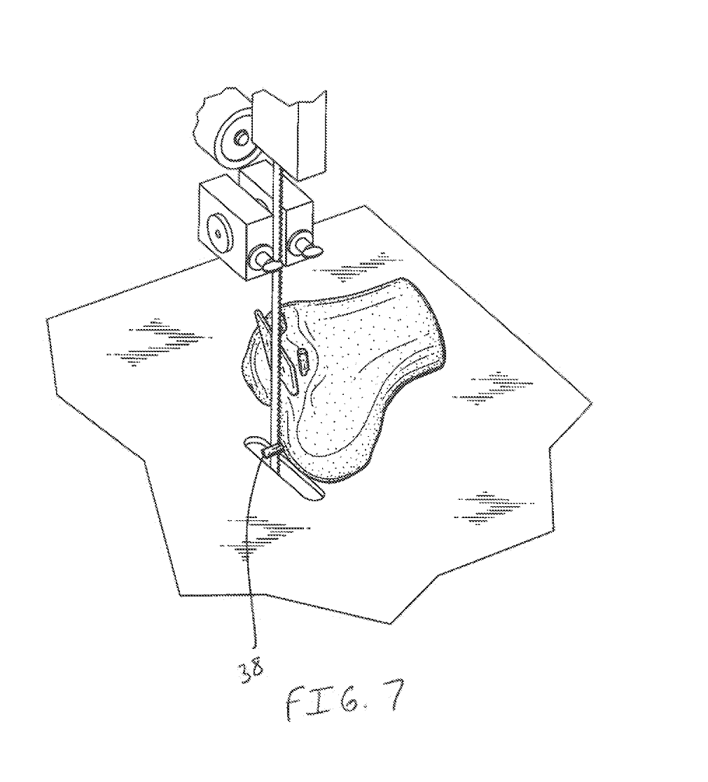

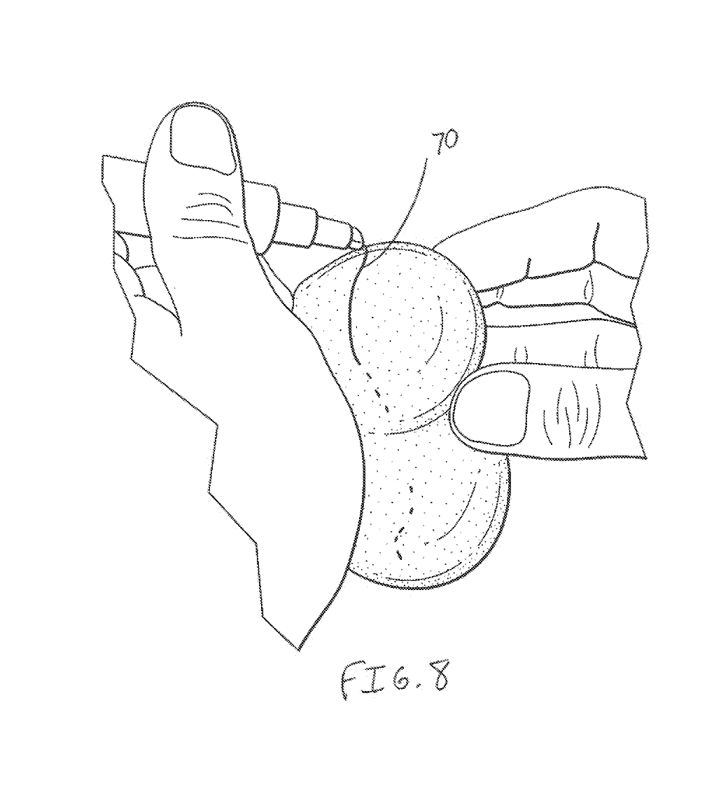

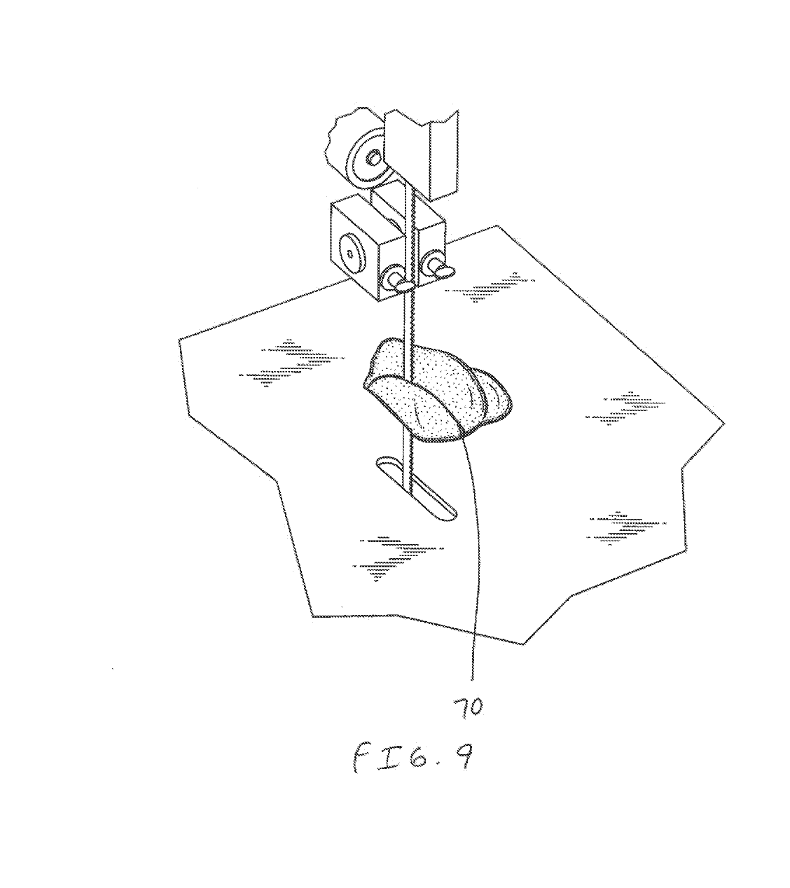

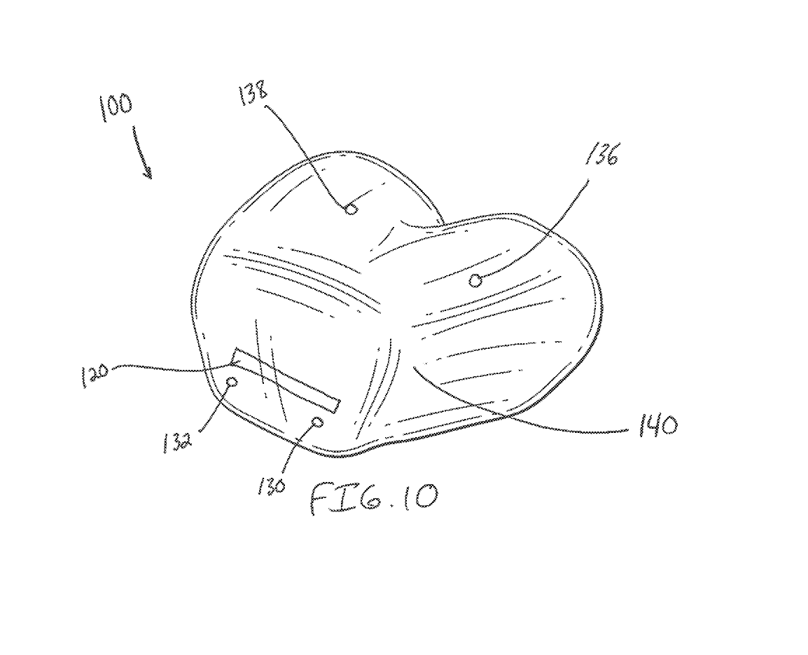

As shown in FIG. 5, while bone model 10 is being rotated by lathe 50, an epoxy 60 including a curing agent and resin is poured on the entire exterior surface of bone model 10. The final coating thickness of the epoxy is preferably several millimeters thick. Once a sufficient thickness of epoxy is achieved, the resin is then cured. Preferably, the resin is a thermo-set resin, UV-curing epoxy, or two-stage epoxy, for example. FIG. 6 represents the resin being fully cured. In one embodiment, the bone model 10 and the cured resin are heated to an appropriate temperature that would allow bone model 10 to melt out of the resin “shell.” In the embodiment shown in FIG. 7, the bone model 10 is being prepared to be removed from the shell which is the custom cutting guide. Second set of two pins 36, 38 are cut from the physical model along the exterior surface of the cured resin. Preferably, the cured resin is cut along an exterior surface thereof at the location of wall 20 and first set of pins 30, 32 in order to ensure that the guide holes are located through the cured resin. Also, these cuts may be made in order to easily remove the custom cutting guide from bone model 10. As shown in FIG. 8, a line may be drawn around the perimeter of the custom cutting guide in order to map out where the guide should be cut in order to remove the guide from physical model 10. Line 70 is a tangent line that would allow the custom cutting guide to easily be removed from the physical model preferably without deforming the custom guide. A tangent line may also be formed on the exterior surface of the updated physical model prior to adding the polymeric composition thereto. The creation of a tangent line may also be done virtually through the use of a mathematical algorithm. As shown in FIG. 9, bone model 10 is then cut along line 70 and is removed from bone model such that custom guide 100 does not surround bone model 10 as shown in FIG. 10.

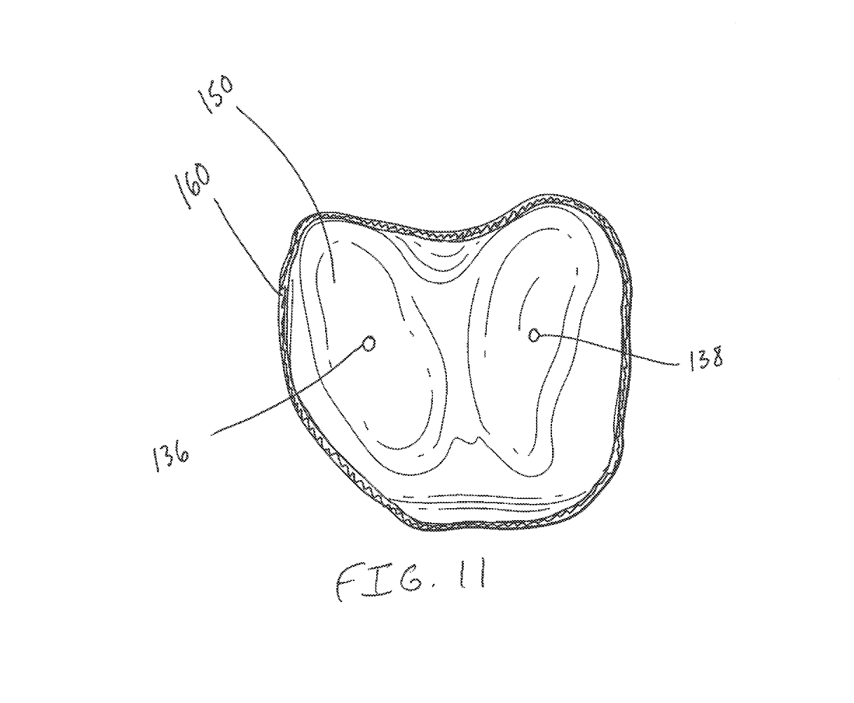

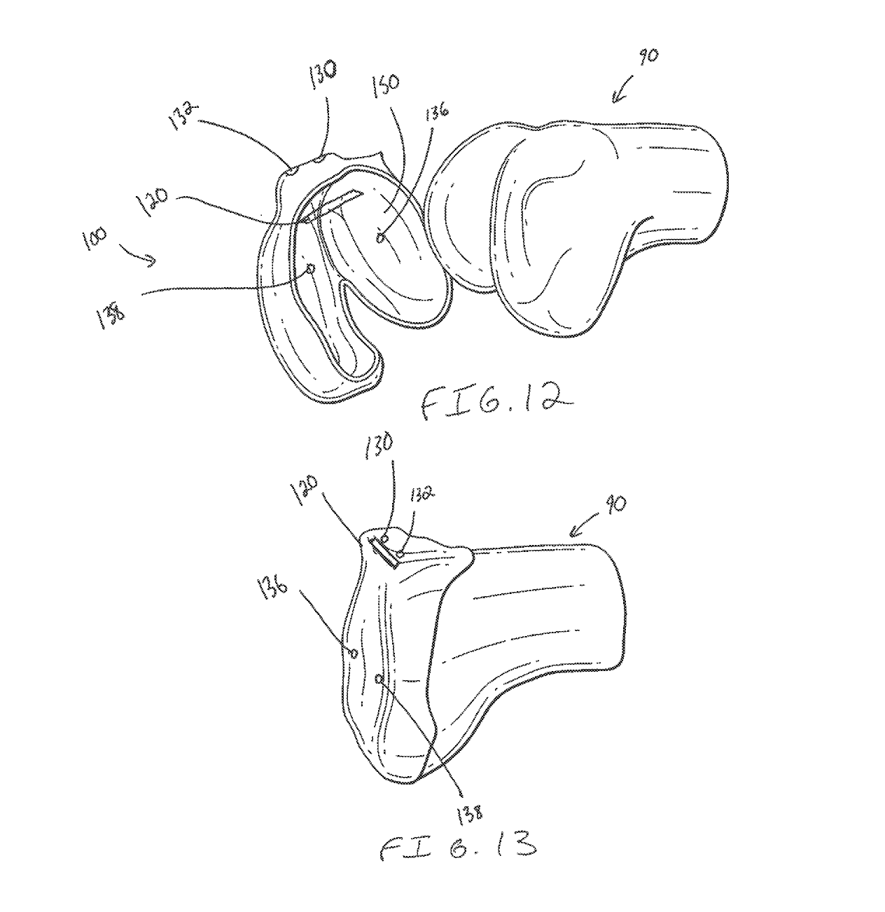

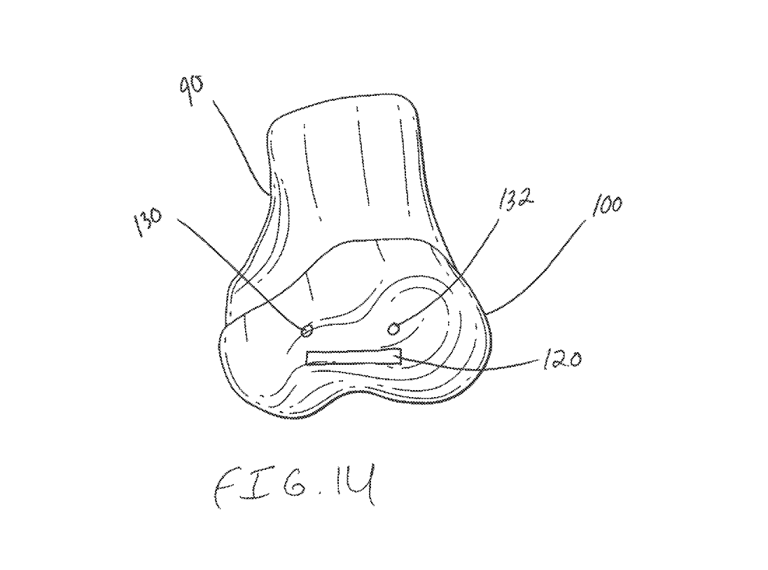

A first set of guide holes 130, 132 are shown in the location where first set of guide pins 30, 32 protruded outwardly from bone model 10. A reference location or guide slot 120 is shown in the location where wall 20 protruded outwardly from bone model 10. Second set of guide holes 136, 138 are shown in the location where second set of guide pins 36, 38 protruded outwardly from bone model 10. While FIG. 10 generally shows an exterior surface 140 of custom guide 100, FIG. 11 generally shows an inner surface 150 of custom guide 100. Custom guide 100 preferably has a thickness of 1 mm to 8 mm as represented by cross-hatching 160 shown in FIG. 11. Second set of guide holes 136, 138 are shown through inner surface 150 of custom guide 100. FIG. 12 shows custom guide 100 just before it is attached on a distal femur 90 of a patient. FIG. 13 represents the custom guide attached to the distal femur.

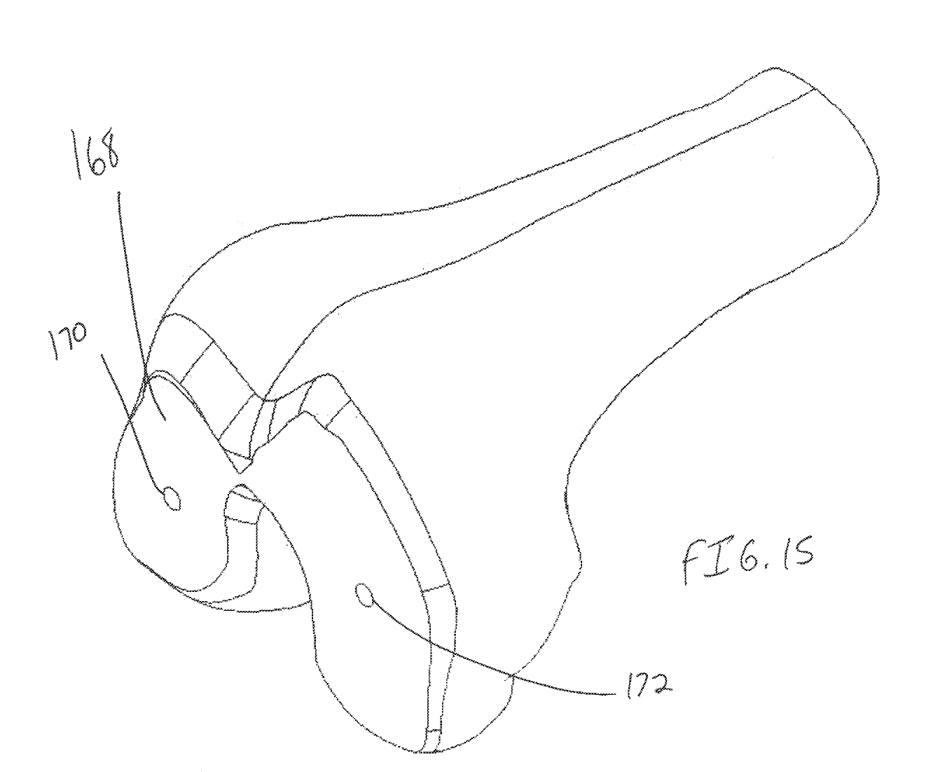

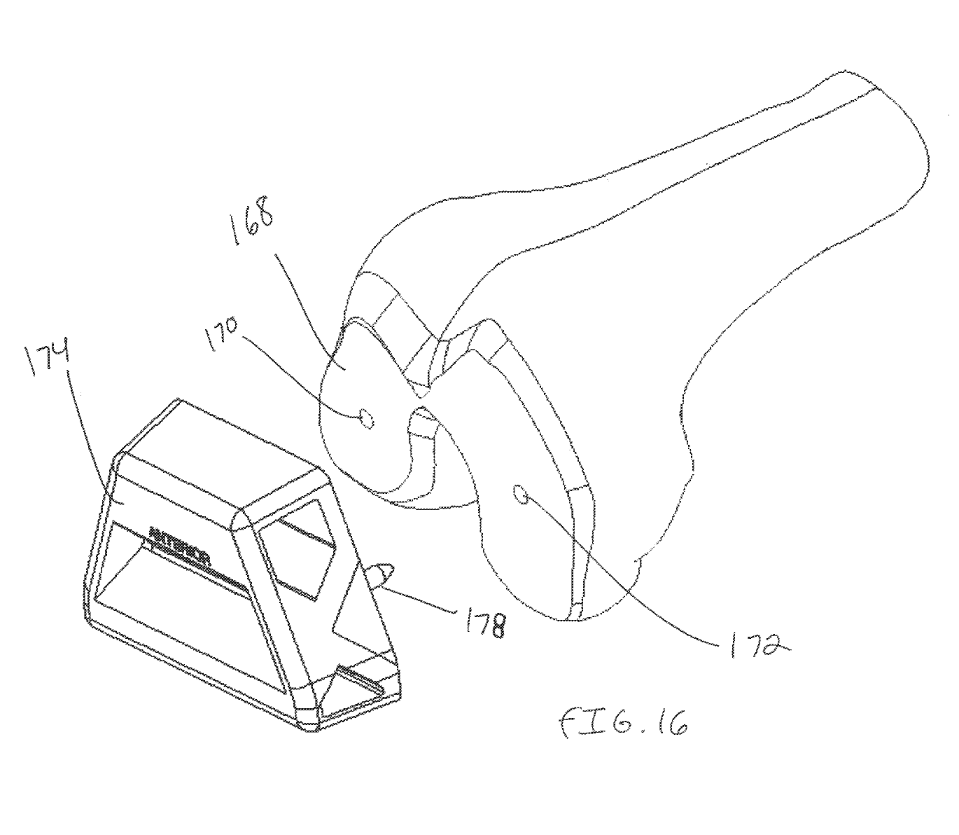

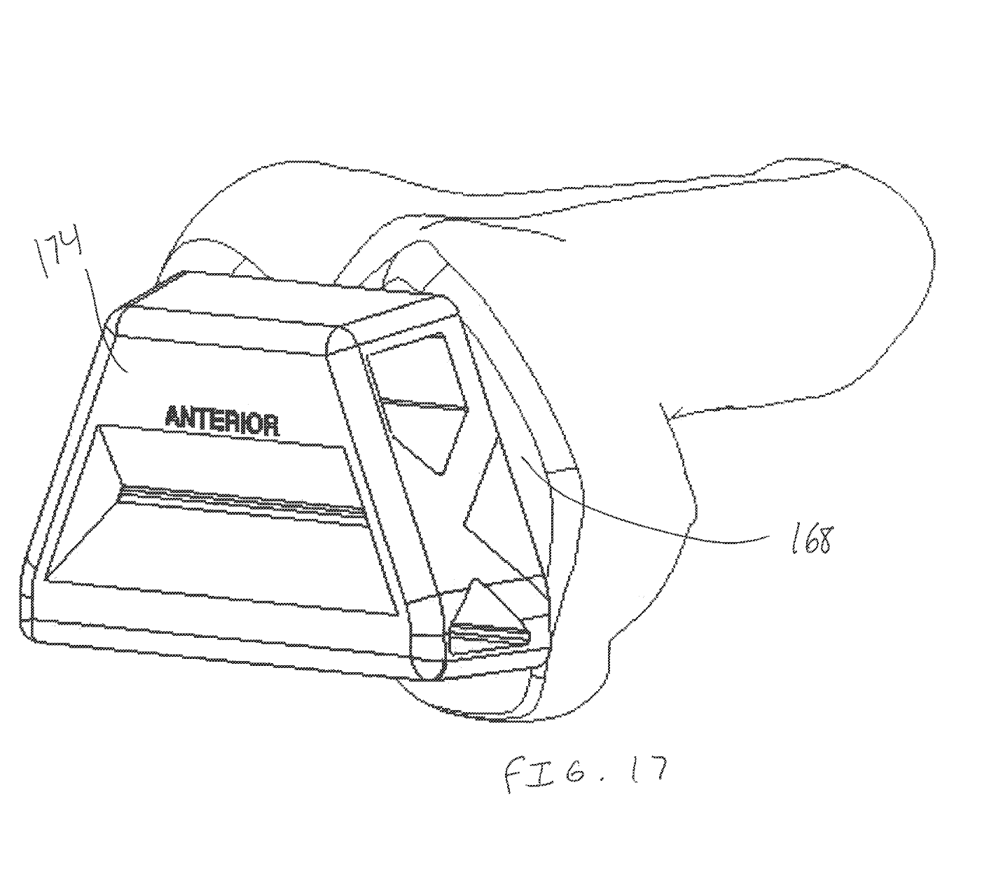

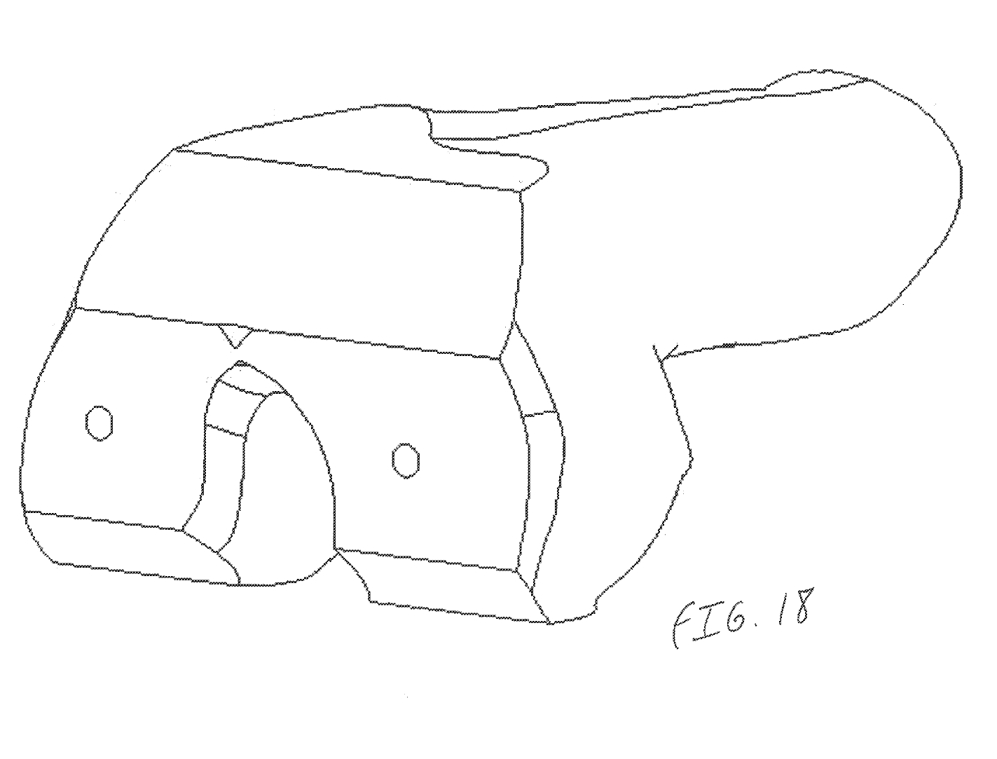

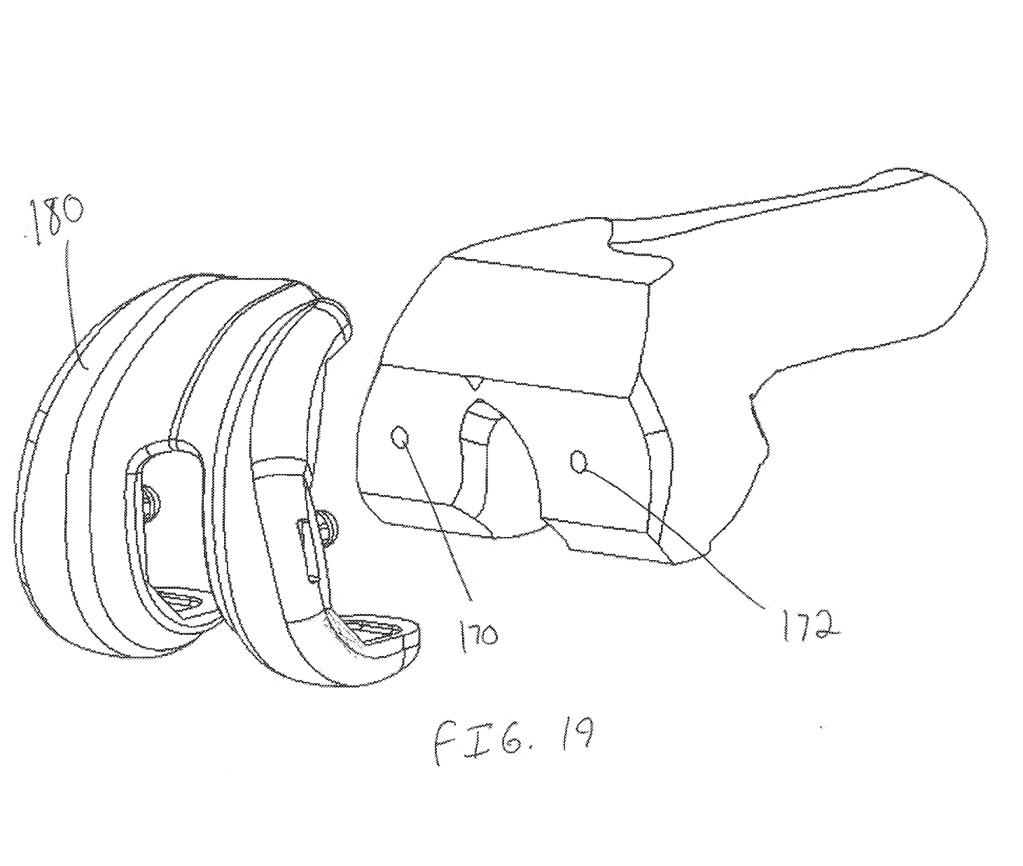

Once custom guide 100 is located in position on the patient's femur as shown in FIG. 14, guide pins may be inserted through first set of guide holes 130, 132 in order to secure custom guide 100 on the patient's femur. An oscillating saw or cutting blade may then be inserted through cutting slot 120 in a posterior direction in order to resect bone located distally of distal resection plane 34. A drill may then be inserted in each second set of guide holes 136, 138 in order to prepare holes 170, 172 on the resected distal surface 168 of the femur as shown in FIG. 15. Holes 170, 172 house guide pins 178 of a four-in-one cutting block 174 after custom guide 100 is removed from the femur and four-in-one cutting block 174 is engaged to the resected bone on the distal surface 168 of the resected femur as shown in FIGS. 16-17 After the anterior, anterior chamfer, posterior chamfer, and posterior cuts are made using the four-in-one cutting block, the femur is fully resected as shown in FIG. 18 and ready to receive a corresponding prosthesis 180 as shown in FIG. 19 that was previously selected by the surgeon.

Although the invention herein has been described with reference to particular embodiments, it is to be understood that these embodiments are merely illustrative of the principles and applications of the present invention. For example, the principles of the present invention are applicable to the following surgeries: Primary Total Knee, Revision Total Knee, Uni-compartmental knee, Patella-femoral, Bi-compartmental knee, Defect Filling/Local Resurfacing in a knee, High Tibial Osteotomy, Primary Hip Replacement, Revision Hip Replacement, Hip Resurfacing, Acetabular Placement, Total Ankle Replacement, Talar Replacement, Talar Resurfacing, Total Shoulder, Humeral Head Resurfacing, Glenoid Resurfacing, Total Elbow, Shoulder Revision, Radial Head Replacement, Wrist, Peri-acetabular Replacement, Distal/Proximal/Total Femoral Replacement, Proximal Tibial Replacement, Distal/Proximal/Total Humeral Replacement, Spinal surgery and surgery to repair trauma. It is therefore to be understood that numerous modifications may be made to the illustrative embodiments and that other arrangements may be devised without departing from the spirit and scope of the present invention as defined by the appended claims.

1. A method of creating a patient specific femoral cutting guide, comprising:
obtaining image data defining the geometry of a patient's femur to create a virtual model of at least a portion of the patient's femur, the virtual model created by the image data being displayed on a computer screen;
selecting at least one set of two first reference locations on the virtual model each representing the location of a guide hole on the patient specific cutting guide;
selecting a location of a reference plane on the virtual model representing a location of an inner wall partially bounding a cutting slot on the patient specific cutting guide;
creating an updated virtual model by:
adding to the virtual model at least two protrusions extending outwardly from the virtual model at the selected location of the at least one set of the reference locations; and
adding to the virtual model a thin wall extending outwardly from the virtual model in an anterior direction from the selected location of the reference plane;
creating a physical model of the updated virtual model including first and second pins extending outwardly from an outer surface of the physical model, wherein the first and second pins are defined by the at least two protrusions extending outwardly from the virtual model, and a wall extending outwardly from the outer surface of the physical model, wherein the wall is defined by the thin wall extending outwardly from the virtual model;
covering the physical model with a polymeric composition; and
allowing the polymeric composition to harden to form the patient specific cutting guide.

2. The method of claim 1, further comprising:
removing the physical model from the patient specific cutting guide by one of a group consisting of melting, cutting, and pulling the physical model.

3. The method of claim 1, further comprising:
cutting the patient specific cutting guide at the locations of the first and second pins and wall of the physical model.

4. The method of claim 3, further comprising:
cutting the hardened polymeric composition along a tangent line such that the physical model may be removed from the hardened polymeric composition.

5. The method claim 1, wherein the patient specific cutting guide has an inner surface that conforms to an exterior surface of the patient's femur.

6. The method of claim 1, wherein the patient specific cutting guide has an inner surface having an infinite number of contact points with an exterior surface of the patient's femur.

7. The method of claim 1, further comprising:
placing a thin metallic material around a circumference of the first and second pins and wall of the physical model.

8. A method of creating a patient specific femoral cutting guide, comprising:
obtaining image data defining the geometry of a patient's femur to create a virtual model of at least a portion of the patient's femur, the virtual model created by the image data being displayed on a computer screen;
selecting anatomical landmarks on the virtual model, the anatomical landmarks used to locate two first reference locations each representing the location of a guide hole of the patient specific cutting guide and a reference plane representing the location of a posterior portion of a cutting slot of the patient specific cutting guide;
creating an updated virtual model by:
adding to the virtual model a protrusion extending outwardly from the virtual model at the selected location of each of the two first reference locations; and
adding to the virtual model a thin wall extending outwardly from the virtual model in an anterior direction from the selected location of the posterior portion of the reference plane;
creating a physical model of the updated virtual model including first and second pins extending outwardly from an outer surface of the physical model, wherein the first and second pins are defined by the protrusion extending outwardly from the virtual model at the selected location of each of the first two reference locations, and a wall extending outwardly from the outer surface of the physical model, wherein the wall is defined by the thin wall extending outwardly from the virtual model in the anterior direction from the selected location of the posterior portion of the reference plane;
covering the physical model with a polymeric composition; and
allowing the polymeric composition to harden to form the patient specific cutting guide.

9. The method of claim 8, further comprising:
cutting the patient specific cutting guide at the locations of the first and second pins and wall of the physical model.

10. The method of claim 9, further comprising:
cutting the hardened polymeric composition along a tangent line such that the physical model may be removed from the hardened polymeric composition.

11. The method of claim 8, wherein the patient specific cutting guide has an inner surface having an infinite number of contact points with an exterior surface of the patient's femur.

12. The method of claim 8, further comprising:
placing a thin metallic material around a circumference of the first and second pins and wall of the physical model.

13. The method of claim 8, further comprising:
selecting additional anatomical landmarks on the virtual model, the additional anatomical landmarks used to locate two second reference locations each representing the location of a guide hole on the patient specific cutting guide.

14. The method of claim 13, wherein the two first reference locations lie along a first plane and the two second reference locations lie along a second plane, and wherein the first plane is perpendicular to the second plane.

15. The method of claim 14, wherein the reference plane of the wall is substantially parallel to one of the first and second planes.
